(19) United States
(12) Patent Application Publication
(10) Pub. No.: US 2008/0167605 A1
(43) Pub. Date: Jul. 10, 2008

(54) Breast Pump

(76) Inventor: Aasmund Torvik, Fosnavaag (NO)

Correspondence Address: DENNISON, SCHULTZ & MACDONALD, 1727 KING STREET, SUITE 105, ALEXANDRIA, VA, 22314, US

(21) Appl. No.: 11/813,491

(22) PCT Filed: Jan. 27, 2006
(86) PCT No.: PCT/NO2006/000039

§ 371 Date: Sep. 17, 2007

(30) Foreign Application Priority Data
Jan. 28, 2005 (NO) 20050494

(51) Int. Cl. A61M 1/06

(52) U.S. Cl. 604/74

(57) ABSTRACT

Breast pump with a suction funnel (2) for resting against a breast, which suction funnel is connected to a pump unit in the form of a flexible receptacle (4) which after compressing and releasing generates a negative pressure in the suction funnel (2) creating a pump action. The suction funnel and the flexible receptacle (4) being connected with a connector part (3). It comprises a release valve (11) at or in the connector part (3), which is provided to release air from the flexible receptacle (4) when this is compressed, the flexible receptacle being bottleshaped.

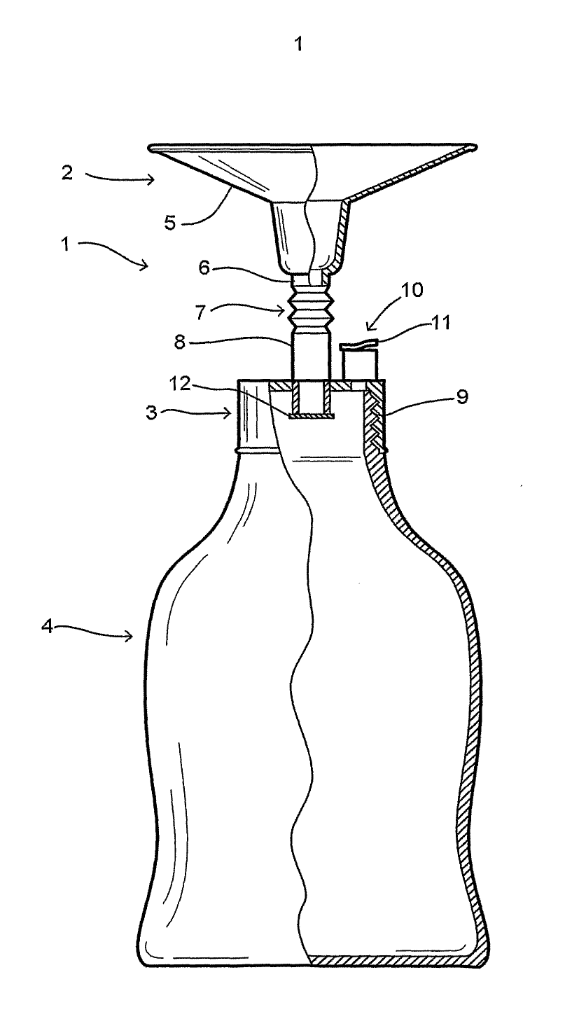

The invention relates to a breast pump for nursing women, as stated in the introductory part of claim 1.

### BACKGROUND

Nursing women will occasionally need a breast pump for pumping breast milk. This may be the case when the women are deprived contact with the child, in cases of illness or when the child has problems with breast feeding. Different breast pumps are known, some band operated and some motor powered.

Some of the existing breast pumps are relatively complicated and expensive. Through rinsing between each use is also required, in some cases requiring time consuming disassembling of the pump.

Already in 1910 Howard V Brumley from New York (U.S. Pat. No. 949,278) proposed to provide threads at the outlet of a sucking funnel to screw on a combined pumping bulb and a collecting receptacle. Even if this did provide a simple solution with few parts, the breast pump of Brumley was not satisfying. It created mess around the sucking funnel. This principle is today used in a modified form for draining pumps, without using the pumped milk, e.g. in a simple PIGEON breast pump from Japan.

Karon Viers from Michigan proposed in 1987 (U.S. Pat. No. 4,705,504) to enter a collecting bag over a corresponding suction member with a one way emptying valve in the bottom. This will still create mess at the suction member and will not provide a suitable collecting.

At the PIGEON breast pump 16200 from Japan, a pumping system is operated with a handle. This pump is complicated and bulky.

In U.S. Pat. No. 4,680,028 (1978) it is proposed to connect a bottle at the connecting tube between a suction member and a bulb pump, to collect breast milk in a convenient and hygienic way. This increases the Lumber of parts to handle and to clean. Additionally, this breast pump requires two handed operation, rendering the use inconvenient.

### Purpose

The main purpose of the invention is to provide a breast pump which is simple and inexpensive to manufacture, efficient to use and easy to clean. This imply that there should be few part which are easy to clean and easy to keep and transport.

Further it is important that it is convenient to use, both by being single-handed and by creating least possible leakage.

It is a particular purpose to provide a breast pump with low dimensions and low price, suitable for distributing in grocery stores, at gas stations, hotel receptions etc.

### The Invention

The purpose is achieved by a breast pump according to the invention, as stated in claim 1. Preferred embodiments of the invention are defined in the dependent claims.

The invention may comprise both breast pumps with an accompanying receptacle and breast pumps without such receptacle, for adding such receptacle as described in the example.

### EXAMPLE

In the following an example of an embodiment shown in the FIGURE is described. The FIGURE shows a partly sectioned side view of a breast pump according to the invention, arranged on an elastic bottleshaped receptacle.

The FIGURE show an embodiment of a breast pump 1 comprising a suction funnel 2, a connection part 3 and a flexible bottleshaped receptacle 4.

The suction funnel 2 has a funnel-shaped contact member 5 being adapted in a manner known per se to contact the breast of a nursing woman. The narrow end 6 of the contact member 5 merges into a flexible intermediate part 7 which is integrated with the connection part 3 over a tubular part 8. The flexible intermediate part 7 allows positioning of the suction funnel 2 relatively to the connector element, with a maximum angle of 90°. The intermediate part 7 is flexible without being elastic, with a threshold twisting moment, keeping it in a chosen position.

The connection part 3 of the example is designed as a screw cap 9 for a bottleshaped receptacle. In addition, it serves as carrier for a tubular valve casing 10 extending beside the tubular part 8 connecting the suction funnel 2 with the connection part 3.

The tubular valve housing 10 connects the interior of the connection part 3 and the ambient air, and has an elastic flap or wing 11 at the end, which provides the valve body of a first one way valve and which can release air from the bottleshaped receptacle 4, without letting it back. The valve body 11 is central to the breast pump 1 according to the invention, as will be described in the following.

The tubular part 8 connecting the suction funnel 3 with the connection part 3 is extended into the screw cap 9 and closed by a second elastic flap or wing 12 with corresponding valve function as the valve body 11. It is provided to release breast milk from the suction funnel 2 into the connection part 2 and from there down into the bottleshaped receptacle 4, and to prevent air from being forced into the suction funnel.

The bottleshaped receptacle 4 of the example is shown with a screw neck to be screwed closely to the screw cap 9 of the connection part 3. The connection between the connection part 3 and the bottleshaped receptacle 4 may be alternatively designed.

The bottleshaped receptacle 4 is manufactured of an elastically compressible material, to revert to its original distended state after being compressed and released of the compression. Due to the first valve body 11, this will suck air from the suction funnel 2. Because this is kept close to the area around the nipple and do not allow entrance of air from the surroundings, a suction effect pumping milk from the breast will occur.

The bottleshaped receptacle 4 may be manufactured particularly for, the purpose and be delivered as a part of the complete breast pump. But it is also possible by choosing design and dimension of the threads of the screw cap 9 to enable connection to a standardized consumer bottle. Some such bottles have proved suitable for a breast pump according to the invention. This makes the transport and keeping of the breast pump easier. The suitable consumer bottles can be soft drink bottles available at almost everywhere in shops, inns, etc.

### Use

An overview of the function of the elements of the shown breast pump is provided. In the following this is examined to show how the breast pump is used.

When the bottleshaped receptacle 4 is closely connected to the connector part 3, the compressing of the receptacle 4 will reduce its volume, to release air passing the first valve body 11.

On releasing the compression of the receptacle 4, the elasticity of the receptacle will press its wall to the original position, to create a negative pressure in the receptacle. Due to this negative pressure, the first valve body 11 will close and the second valve body 12 will open and the negative pressure of the receptacle will act on the breast. By compressing and releasing the receptacle 4 repeatedly, one will thus be able to pump breast mill will the breast pump, the first and the second valve bodies 11, 12 opens and closes, respectively closes and opens. Breast milk pumped in this way will flow through the connector part 3 down into the bottleshaped receptacle and be collected there.

### MODIFICATIONS

The valve bodies 11 and 12, which can have other designs than shown, may be incorporated in a common valve casing being a integrated part of the connector part 3. As mentioned, the breast pump can function with only the first valve body 11 releasing air when compressing the bottleshaped receptacle.

1. Breast pump with a suction funnel (2) for resting against a breast, which suction funnel is connected to a pump unit in the form of a flexible receptacle (4) which after compressing and releasing generates a negative pressure in the suction funnel (2) creating a pump action, said suction funnel (2) and the flexible receptacle (4) being connected with a connector part (3), characterized in that it comprises a release valve (11) at or in the connector part (3), which is provided to release air from the flexible receptacle (4) when this is compressed, the flexible receptacle being bottleshaped.

2. Breast pump according to claim 1, characterized in that it is provided for delivery without the flexible receptacle, this being provided at a place of use.

3. Breast pump according to claim 2, characterized in that the connector part (3) comprises a screw cap (9) for close attaching of a threaded bottleneck, preferably for a standardized consumer bottle with an elastic flexible wall.

4. Breast pump according to one of the claims 1 to 3, characterized in that it comprises a second valve (12) in the connection to the suction funnel (2), which is provided to close for entrance of air to the suction funnel, and keep open for milk there from.

5. Breast pump according to one of the claims 1 til 4, characterized by comprising a flexible intermediate part (7) between the suction funnel (2) and the connector element (3), which is provided to allow the bottleshaped receptacle (4) a convenient position of use.
